(19) United States
(12) Patent Application Publication
(10) Pub. No.: US 2014/0216305 A1
(43) Pub. Date: Aug. 7, 2014

(54) Adjustable Bi-Level Surgical Accessory Table

(71) Applicants: Scott D. Hodges, Oultewah, TN (US); Christopher B. Young, Chattanooga, TN (US); Ira Bruce Young, Chattanooga, TN (US)

(72) Inventors: Scott D. Hodges, Oultewah, TN (US); Christopher B. Young, Chattanooga, TN (US); Ira Bruce Young, Chattanooga, TN (US)

(21) Appl. No.: 14/247,223

(22) Filed: Apr. 7, 2014

Related U.S. Application Data
(63) Continuation-in-part of application No. 13/155,223, filed on Jun. 7, 2011, now Pat. No. 8,689,704.
Application No. 13/155,223 is a continuation-in-part of application No. 11/948,089, filed on Nov. 30, 2007, now abandoned.
Application No. 11/948,089 is a continuation-in-part of application No. 11/436,609, filed on May 19, 2006, now abandoned.
(60) Provisional application No. 60/682,851, filed on May 20, 2005.

(51) Int. Cl. A61B 19/02

(52) U.S. Cl. CPC A61B 19/0248; A61B 2019/025; A61B 2019/0255
USPC 108/3

(57) ABSTRACT

A sterilizable surgical accessory table includes a pair of independently movable tabletops supported by a base. The front tabletop is supported by a carriage which allows the position of the front tabletop to be adjusted fore and aft, while maintaining it in a horizontal plane. The rear tabletop move fore and aft, and can also be tilted about a transverse horizontal axis. The tabletops can be raised or lowered in unison when a release pedal near the bottom of the base is depressed. Each tabletop can be independently moved fore and aft when a release bar at the front of the tabletop is moved to a release position, and the rear tabletop can be tilted when release levers below the tabletop are lifted to a release position.

This application is a continuation-in-part of copending application Ser. No. 13/155223, filed Jun. 7, 2011, which was a continuation-in-part of application Ser. No. 11/948,089 filed Nov. 11, 2007, now abandoned, which was a continuation-in-part of application Ser. No.11/436,609 filed May 19, 2006, now abandoned, which claimed benefit of provisional Application No. 60/682,851 filed May 20, 2005. The contents of the above applications are incorporated herein by reference.

### BACKGROUND OF THE INVENTION

The invention relates generally to medical tables and, more particularly, to an improved bi-level medical table that is compact, adjustable, and easily draped.

Various types of specialized equipment are used to perform surgeries and other medical procedures. In particular, stainless-steel tables found in doctor's offices and operating rooms are used to hold a wide variety of sterilized medical instruments and supplies during surgical or other medical procedures.

Such tables arose from the need to place medical instruments and accessories within easy reach of doctors and nurses during medical or surgical procedures, and from requirements that medical instruments and supplies, as well as work surfaces of the table itself, be sterilized prior to use and kept sterile until used.

For example, many hospital regulations require routine sterilization of all parts of medical tables used in surgery that are at about thirty inches or more above the floor of an operating room. As another example, the Centers For Disease Control and various medical organizations strongly recommend the routine sterilization of medical instruments, examination tables, and other medical equipment. Because most conventional surgical tables are not easily disassembled for sterilization, the sterilization requirements are typically met by laying a sterilized disposable drape (cover) on the table surface(s) prior to the start of a medical procedure and then disposing of the drape(s) after the procedure is completed. In contrast, medical instruments and supplies are typically sterilized in an autoclave located in the hospital or other healthcare facility. Examples of acceptable instrument sterilization methods include steam under pressure, dry heat, chemical vapor, radiation, cold sterilization, or ethylene oxide gas.

Conventional surgical tables are typically made from surgical grade stainless steel and their size makes them bulky and awkward to use. Such tables generally have two or more horizontal shelves stacked one above the other and attached to four upright corner posts. The edges of the shelves and other parts of the tables are typically rounded to avoid snagging or tearing a sterilized disposable surgical drape that is laid over a particular shelf or shelves. To provide mobility, such tables are commonly equipped with an anti-static wheel at each bottom corner. The lower and upper shelves of conventional tables are structured to hold instrument sterilization trays and baskets, lap trays, scope trays, plastic baskets, wire baskets, packaged medical supplies and accessories, and other health care products.

The sizes of conventional tables vary, but a typical surgical table measures about seventy-two inches wide, about thirty-four inches high, and about twenty-four inches deep, and has a unibody construction that prevents sterilizing the table (or its components) in standard hospital autoclaves, which typically measure about three feet wide, about six feet tall, and about five feet deep. Thus, conventional surgical tables used in the operating rooms of most hospitals cannot be readily sterilized using steam under pressure, dry heat, chemical vapor, or other types of sterilization methods. Consequently, the shelves of conventional surgical tables are covered with sterile disposable surgical drapes prior to use. A typical use of a conventional surgical table is described below.

Prior to a surgery, a nurse or technician will prepare the operating room. This is typically accomplished by cleaning the surgical table using a hand-scrubbing method and/or an ultrasonic cleaning method. Once the table is cleaned, the nurse covers the table's work surface(s) with a sterilized disposable surgical drape (or drapes). After the drape(s) have been properly positioned, the nurse loads previously sterilized medical instruments and/or supplies directly onto the surgical drape(s). Generally, the instruments themselves will have been removed from the hospital autoclave a short time before; however, sterilized pre-packaged instruments and/or supplies may be loaded onto the drapes as well. Typical medical instruments and/or supplies may include kits of surgical instruments and medical devices and materials appropriate to the particular surgery that will be performed, as well as autoclave instrument sterilization trays and other sterilization receptacles. Depending on the medical procedure involved, it is not uncommon for a six foot long conventional surgical table to be covered from end to end (and front to back) with sterilized instruments and/or supplies by the time the nurse completes the preparations.

During surgery, the nurse is typically positioned between the doctor and the surgical table. From this position, the nurse can retrieve sterilized instruments and/or supplies and hand them to the doctor as necessary. The nurse can also take contaminated instruments from the doctor and place them on the surgical drape or in sterilization receptacles that were pre-positioned on the surgical table. Because most conventional surgical tables are about six feet long, the nurse often has to move back and forth from one end of the table to the other in order to retrieve (or set down) a sterilized or contaminated item. The size of the table hinders free and easy movement of the table and the people in and around the operating room. Small operating rooms further exacerbate this problem, because the bulky dimensions of a conventional surgical table leaves the nurse (and/or doctor(s)) with little floor space in which to work. Additionally, instruments and/or drapes positioned on the table may fall to the floor if the table is bumped or a drape is snagged. After the surgery or other medical procedure is finished, the drapes are considered to be biohazards, and must be properly incinerated, which increases disposal costs and adds regulatory compliance.

Health care equipment suppliers have proposed numerous approaches for configuring surgical tables and methods for sterilizing them. For instance, one conventional configuration solution adds a top shelf to the table. This top shelf is as long as the table top, but slightly narrower. Although the plane of the top shelf is not adjustable relative to the plane of the table top, the top shelf may be raised and lowered between heights of about twelve inches to about fifteen inches above the table top. This adjustability feature allows persons of about five feet four inches or less to reach instruments on the top shelf without having to use step stools. However, this conventional approach suffers drawbacks in that in addition to traversing the six foot length of the table, the nurse may also have to tilt the trays or baskets on the top shelf and/or stand on tiptoe or on a step stool) to see what instruments and/or supplies are inside. Moreover, instrument trays and other supplies are likely to fall off the narrow top shelf and cascade onto those on the table top. Additionally, use of the top shelf requires an extra disposable drape, which increases the hospital's overhead costs.

Yet another approach, disclosed in U.S. Pat. No. 4,927,214 to Kaufman et al., provides an operating room instrument table assembly formed of a plurality of modular table units. Each table unit includes castor wheel supports, a base frame attached to the castor wheel supports, a table top support frame connected to the base frame, and a removable table top detachably connected to the table top support frame. The removable table top is sterilizable as a unit with the instruments used in a medical procedure. The bare frame includes two upright posts. The table top support frame includes two tabular supports connected by a center connector. Each tabular support is sized to fit over one of the upright posts.

One disadvantage of this operating room instrument table is that it consumes a large area of floor space since only a single table top is included for each movable base/table support frame. Further, if a larger work area is desired, then separate table tops, each on its own movable base, must be disposed side-by-side, and or in a circular configuration, with the surgeon or nurse in the middle. This leads to yet another disadvantage, which is that the person handling the instruments is not free to move about the operating room, but rather is confined within the center of the modular table units. To move about the operating room, the person must push one of the modular table units out of position, and risk bumping another table unit and thereby knocking instruments into disarray and/or onto the floor. Additionally, to access instruments during a medical procedure, the person may be required to move back and forth from one portion of the table to another. During long medical procedures, this may generate fatigue and/or increase the risk that instruments may be bumped and/or dropped. In addition, only the table top is removable for sterilization. The table support frame (e.g. tubular supports connected by a center connector) remains connected to the movable base, and thus is not sterilizable with the removable table top and/or the instruments.

Another approach is disclosed in U.S. Pat. No. 6,189,459 to DeAngelis, which describes a collapsible auxiliary instrument shelf for use in surgical operating rooms. As described in this patent, a conventional surgical table includes a main shelf that is supported at each corner by a leg. A castor is attached to the bottom of each leg. The auxiliary shelf is supported above the main shelf by a pair of posts. Each post is affixed to one of the rear legs that support the main shelf.

One disadvantage of this apparatus is that neither the auxiliary shelf nor the surgical table to which it is attached are detachable for sterilization. Another disadvantage is that paper and/or clear plastic surgical drapes, with all the drawbacks referenced above, must be used to cover the auxiliary shelf and table before the auxiliary shelf and table can be used in surgery. A further disadvantage is that the auxiliary shelf cannot be angled relative to the main shelf. Consequently, it is difficult for a user of the table to view and/or access items placed on the auxiliary shelf.

Yet another proposed approach is disclosed in U.S. Pat. No. 5,551,674 to Boyd, et al., which discloses an accessory tray for use in a surgery, and, more particularly, a sterilizable accessory tray for supporting absorbent material pads for use in neurosurgery. The tray is supported on a conventional surgical stand, which has a movable base, an adjustable support pole attached the base, and a single shelf cantilevered from the top of the support pole. The accessory tray is removable from and in a fixed angular position relative to the shelf of the surgical stand. However, the shelf and other parts of the surgical stand cannot be readily disassembled and sterilized. Preferably, the accessory tray is made of a disposable plastic material, but it may also be made of a sterilizable plastic or metal material.

A disadvantage of the accessory tray of Boyd, et al. is that the tray is particularly adapted to store sterile absorbent pads commonly used in neurosurgery to absorb blood or retain accumulated blood or brain fluids, meaning it has little if any utility for other medical procedures. Although the accessory tray may be removed from the shelf and sterilized, the tray, much like a surgical drape, is preferably removed and thrown away. Consequently, each use of the surgical stand requires either a new sterilized disposable drape or a new sterilized accessory tray. Another disadvantage of this apparatus is that the shelf of the surgical stand is equipped with a continuous raised lip that tends to retain blood and other fluids drained from any used surgical instruments and/or supplies that may be placed on the shelf during a procedure.

Recent medical studies have generated stringent sterilization requirements, most of which are difficult or impossible to satisfy simply by hand cleaning and ultrasonic cleaning alone. In addition to being bulky and non-ergonomic, conventional surgical tables are difficult to sterilize and are costly because they require the use of expensive sterilized disposable drapes.

Thus, there is a need for an improved configuration of a surgical table, particularly a compact table, used during surgery in a hospital operating room, which is easier to sterilize and use in surgery than conventional surgical tables.

### SUMMARY OF THE INVENTION

The invention meets the foregoing needs and avoids the drawbacks and disadvantages of the prior art by providing a compact, ergonomically-designed, medical table for use in surgery and other medical procedures that has components that may be readily disassembled and sterilized in a standard autoclave of the type typically found in hospitals.

A table manufactured according to the invention may be used in surgeries or other medical procedures performed in human health care facilities or in animal health care facilities. Both types of medical facilities may include autoclaves or similar sterilizers.

The table of the invention may be used either to store medical equipment and/or instruments or as a workspace for performing a medical procedure. Users of a table manufactured according to the invention may include, but are not limited to, veterinarians, traditional doctors and surgeons, medical staff, and other persons who work in the animal or the health care industry.

Accordingly, in one aspect of the invention, a modular surgical table has a movable base which supports two removable tabletops. A first sterilizable tabletop structured to hold sterilized medical instruments and supplies during a medical procedure is supported by a pair of uprights at the front of the base. A second sterilizable tabletop is supported by another pair of uprights at the rear of the base. Each of the tabletops is removable so that it can be sterilized separately. Additionally, at least the rear, upper tabletop can be tilted about a horizontal axis, and the tops can be moved up and down in unison as desired by means of a pedal at the bottom of the unit.

### BRIEF DESCRIPTION OF THE DRAWINGS

In the accompanying drawings:

FIG. 1 is a perspective view of a surgical accessory table embodying the invention, from the front.

FIG. 2 is a corresponding view, with its table tops removed.

FIGS. 3a -3e are, respectively, top, front, perspective, side and sectional views of an upper table carriage, shown in its flat or level position.

FIGS. 4a -4e are similar views, showing the carriage in a slightly tilted position.

FIGS. 5a -5e are similar views, showing the carriage tilted a full 10°.

FIGS. 6a -6e are similar views, showing the carriage returned to the level position.

FIGS. 7a -7d are , respectively, top, front, perspective and side sectional views, showing the upper carriage in its full rear position.

FIGS. 8a -8d are similar views, showing a handle, having a locking blade, raised to release the blade from engagement with a slot so that the carriage can be moved fore and aft.

FIGS. 9a -9d are similar views, showing the carriage moved fully forward.

FIGS. 10a -10d are similar views, showing the handle released, with the blade seated in a slot to lock the carriage in place.

FIG. 11 is a perspective view of the bottom of one of the tabletops.

### DETAILED DESCRIPTION OF THE INVENTION

In this description, “front” should be understood to mean the side of the device having the shorter uprights. “Rear” means the side having the longer uprights. “Fore and aft” means from front to rear and vice-versa. “Lateral” means left to right and vice-versa. “Horizontal” herein means geographically horizontal, when the device is standing upright on a level floor.

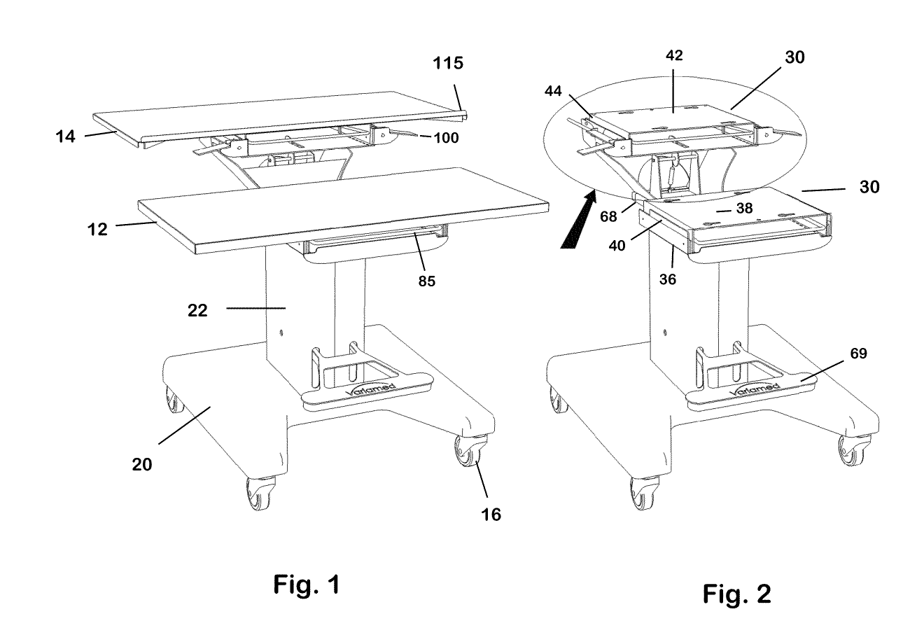

As shown in FIGS. 1 and 2, a surgical accessory table embodying the invention includes a base 10, which supports two independent tabletops 12, 14.

The base includes a rectangular bottom frame 20, which preferably has wheels or casters 16 at four corners. Preferably, at least some of the wheels have a locking mechanism (not shown) to immobilize the table when desired.

Four uprights (not shown) are welded to the frame, near its corners. The uprights are interconnected by a housing 22 which conceals the channel elements while providing a strong interconnection between them.

Horizontal braces (not shown) interconnect the upper ends of the four uprights to rigidify the base.

Each of the tabletops (which have been removed in FIG. 2) is supported by a carriage 30 which permits the tabletop to move fore and aft in a horizontal plane. The carriage is described in detail below.

Each carriage comprises a stationary lower tray 36 and a movable upper tray 38. Ball bearings drawer slides 40 are disposed between the two trays, to eliminate sliding friction.

The upper tray 38 has a flat central portion 42 with downturned flanges 44 at opposite lateral edges. The respective parts of the slides are connected to these flanges by screws or rivets.

The slides 40 support the upper tray and enable it to move unidirectionally along the fore-and-aft horizontal axis, which is parallel to the slides. The carriage body cannot move in any other direction, and cannot yaw, pitch or roll.

Keyhole-shaped apertures 50 (preferably four) are formed in the central portion of the upper tray. The apertures are aligned all facing the same way, with their larger ends to the left, and their lengths running in a lateral direction. The function of the apertures is described further below.

The rear carriage is further provided with the ability to tilt forward and rearward.

All the parts described below are made of heavy gauge stainless steel sheet, unless otherwise noted, and all connections are welds, unless otherwise noted.

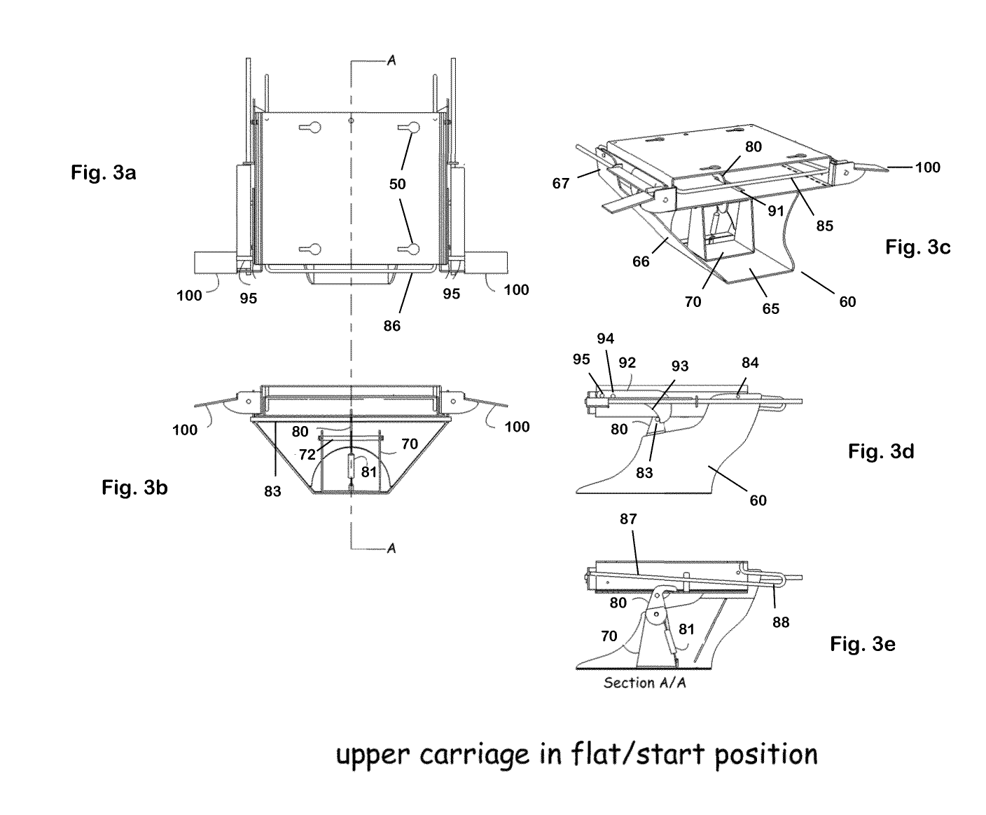

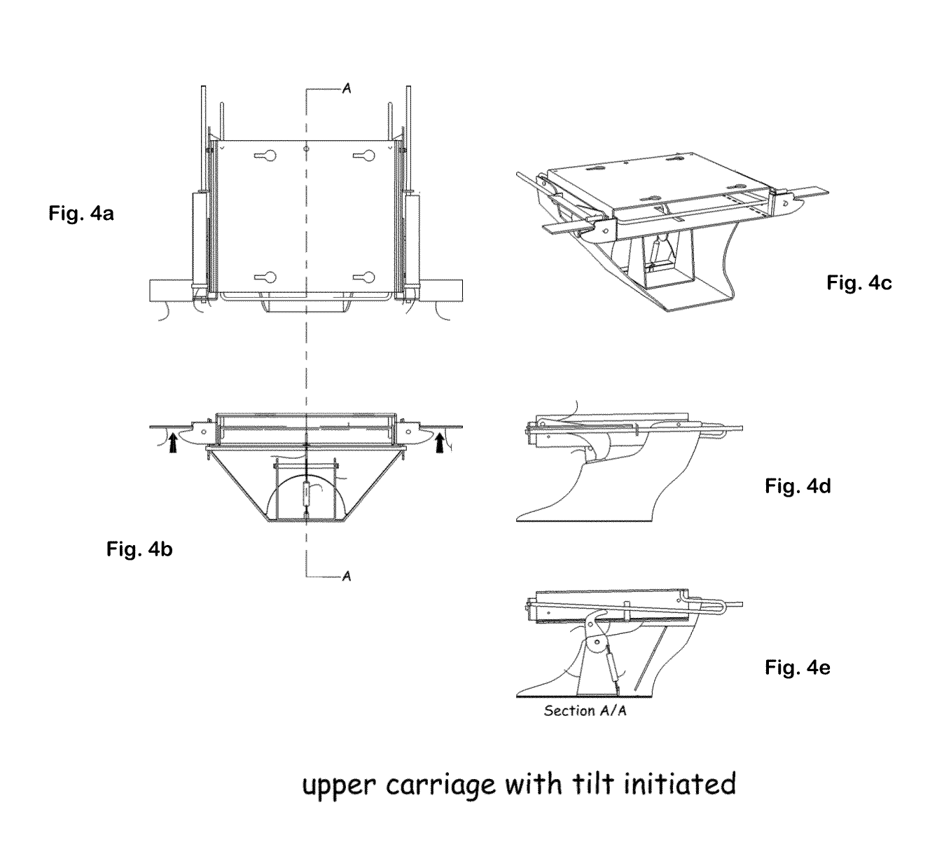

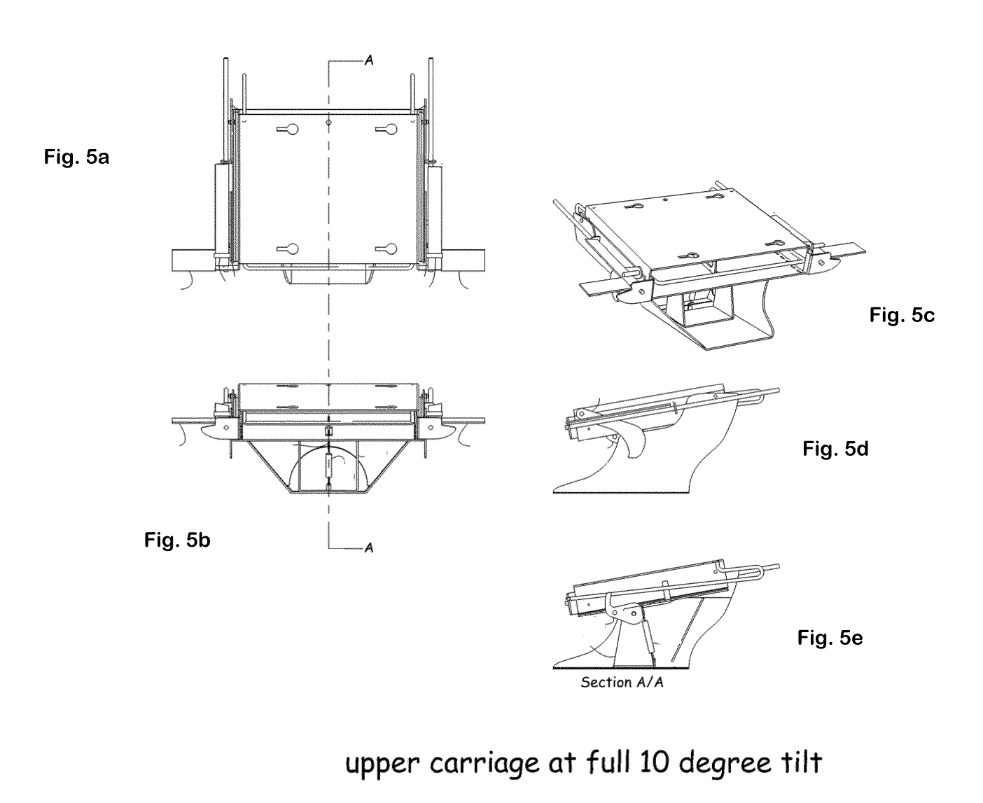

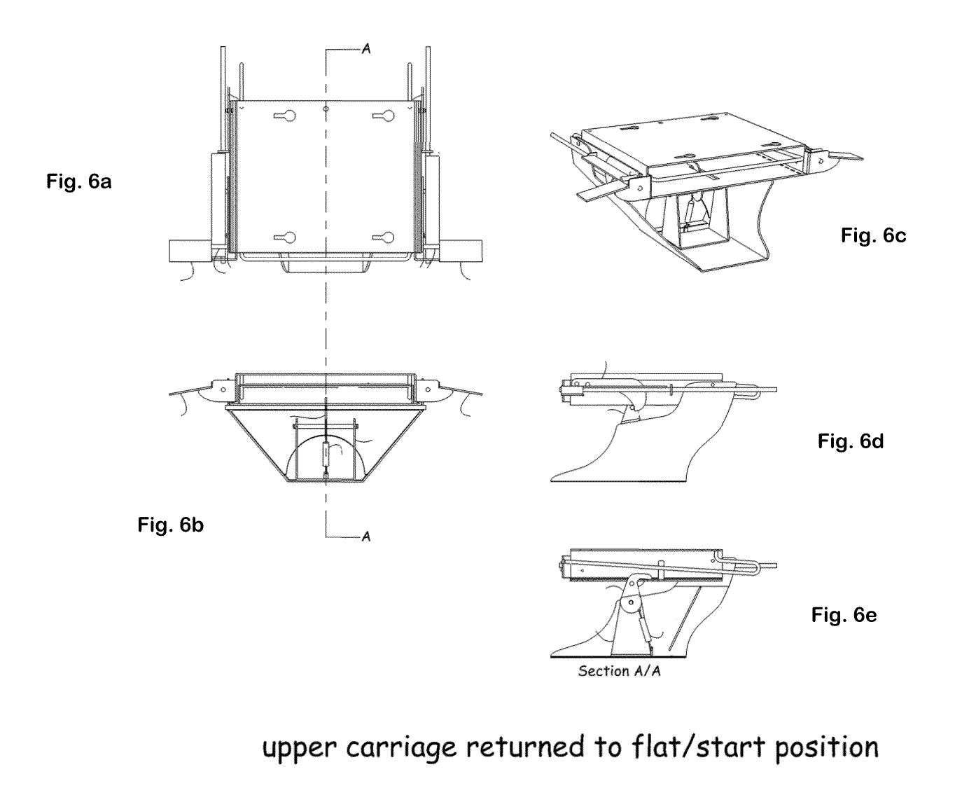

Each carriage is supported by a cradle 60 (e.g., FIG. 3c) formed from a piece of sheet metal which is bent so as to have a horizontal bottom 65, a pair of oblique portions 66 extending upward from either side of the bottom, and parallel vertical portions 67 extending upward from the oblique portions. The cradle is reinforced by two braces (not shown) which are connected between the oblique portions of the base. The cradles are supported by a structure 68 (FIG. 2) which can be moved up or down by means of a hydraulic mechanism (not shown) operated by foot pedal 69 near the bottom of the table. The cradles can be raised by pumping the pedal, or lowered by holding the pedal full down.

A U-shaped bracket 70 is connected to the base, near the center of the bottom portion. The arms of the bracket extend parallel to one another perpendicularly upward from the bottom. A hole is formed near the top of each arm; the two holes are aligned and serve as journals for a pivot rod 72 which passes through them. The ends of the rod have circumferential grooves for receiving respective C-clips outside the arms, to keep the rod centered with respect to the bracket.

A hook member 80 is connected to the pivot rod, which passes through the hook. The hook in biased toward the rear of the table by a tension coil spring 81 which extends between the bottom of the hook and a tang formed at the bottom of the bracket.

A second rod 83 also passes through the hook and is connected to it. The second rod, which supports the table carriage from below, is called the “support rod” hereafter. The support rod is longer than the pivot rod, and is parallel to it.

The lower tray of a sliding mechanism is pivotally connected between the vertical portions of the base by a pair of hinge pins 84. The lower tray rests on the support rod, which can move to change the tilt angle of the tray as described further below.

A handle 85 is provided to enable one to move the upper tray fore and aft, and to lock it in a selected position. The handle is formed from a single metal rod which is bent so as to have a transverse middle segment 86 between two fore-aft segments 87. The free ends of the fore-aft segments are connected to opposite sides of the upper tray, near its rear edge. A hairpin segment 88 near each end increases the handle's flexibility. The exposed transverse segment runs in front of and between the trays. A user may use this portion to push or pull the upper tray horizontally, and may lift it to release two blades 90 (which are connected to the longitudinal segments of the handle) from apertures 91 in which they normally seat. A series of such apertures are formed in the lower tray, so that the user may incrementally adjust the fore-aft position of the support by lifting, and then pushing or pulling, the handle.

As mentioned before, the tilt angle of the upper tray can be changed, from horizontal to a forward tilted angle of about 10°. In its horizontal position, the lower tray is supported from below by the support rod. It is also held down in this position by the hook, which protrudes through an aperture 91 in the lower tray, and engages it from above. The user cannot inadvertently tilt the table, in this position.

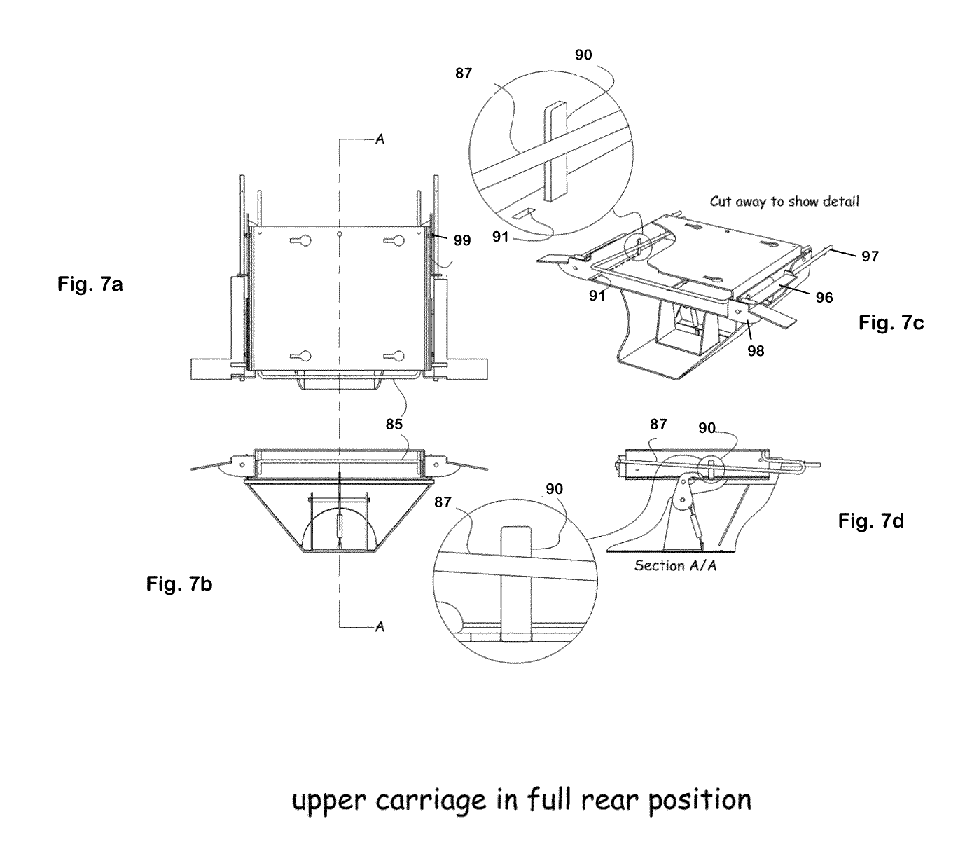

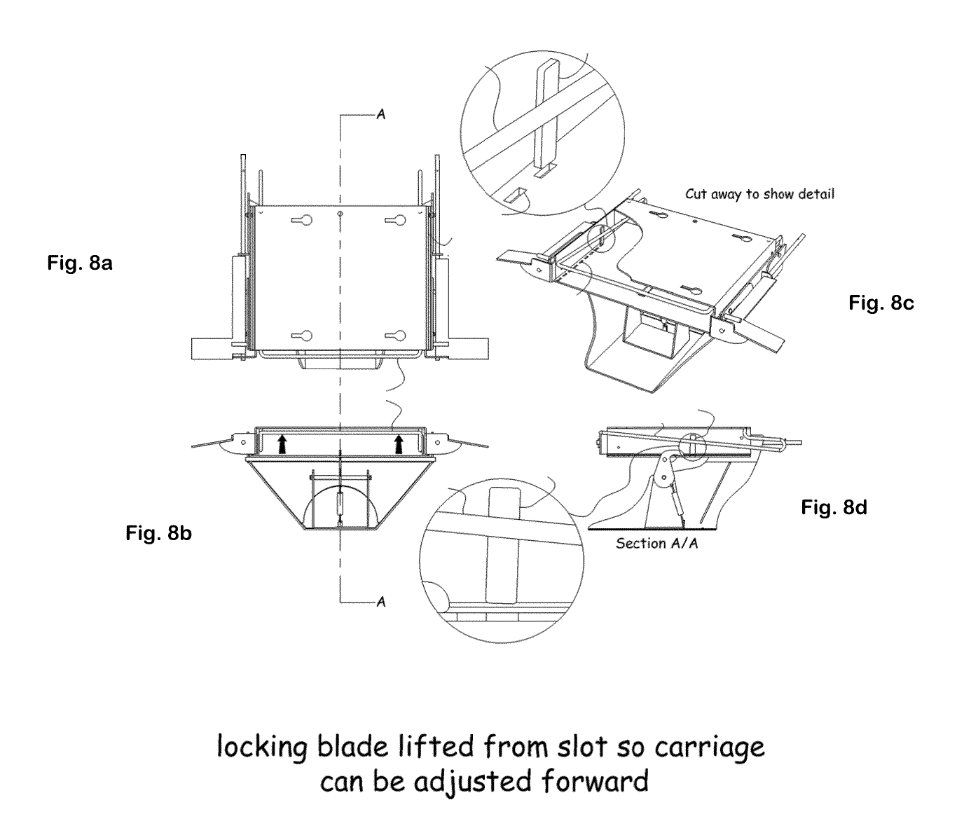

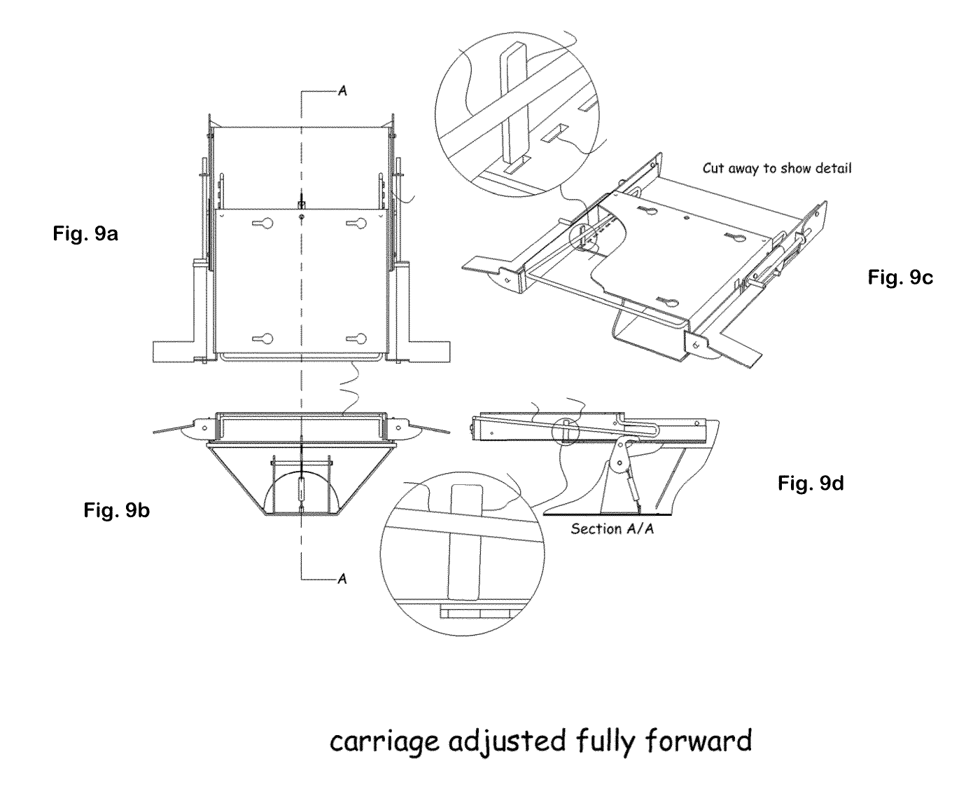

To release the hook from its engaged position, and thus permit tilting of the support, there are two levers 92 (see FIGS. 7a -7d), which are pivotally mounted on the lower tray at opposite sides. The levers have cam surfaces 93 at their rearward ends, which engage the protruding ends of the support rod 83. Each lever has a pivot connection 94 to the lower tray, and a release pin 95 which extends horizontally outward from the lever near its forward end. Lifting the release pin depresses the end of the lever engaging the support rod, whereupon the cam surfaces on the lever press the ends of the support rod down and forward, releasing the hook from its engagement with the lower tray, and simultaneously allowing the lower tray to fall to a lower position at which it is tilted forward toward the user.

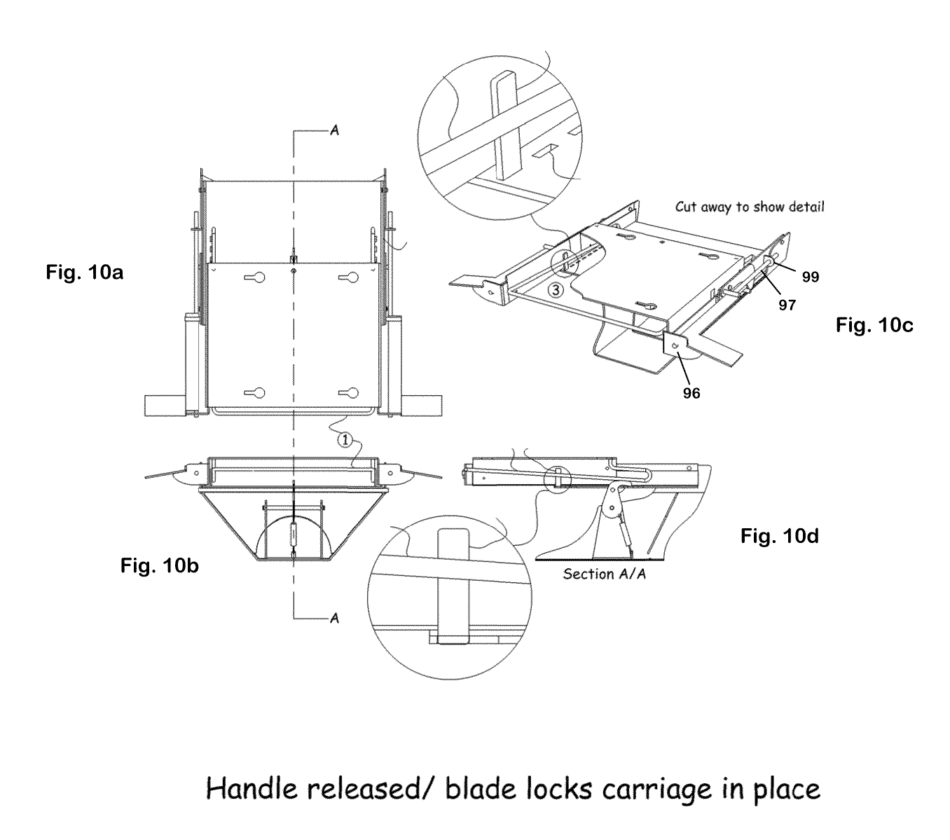

The release pins lie above a pair of L-shaped plates 96 at either side of the support. Each L-shaped plate is connected to a respective rod 97 running fore-and-aft, whose forward end is retained in a hole on a transverse tab 98 which protrudes outward at the front of the upper tray. The rearward end of each rod is supported by an eye 99 (FIG. 10c) connected to the lower tray. The rod can slide and pivot in the eye.

If one lifts the transversely protruding arms or “ears” 100 of the L-shaped plates, the fore-aft arms 101 engage and lift the release pins. Thus the user can release the table, allowing it to fall to its tilted position, simply by lifting on the ears. The table can be manually restored to its horizontal position when desired, whereupon the hook is automatically returned by the tension spring to its locked position.

The tabletops 12, 14 are identical, with one exception: the rear tabletop 14 has an upturned lip 115 (FIG. 1) along its lowermost edge, to prevent small items from rolling off when the tabletop is tilted forward. The corresponding edge 115′ on the front tabletop 12 is turned downward.

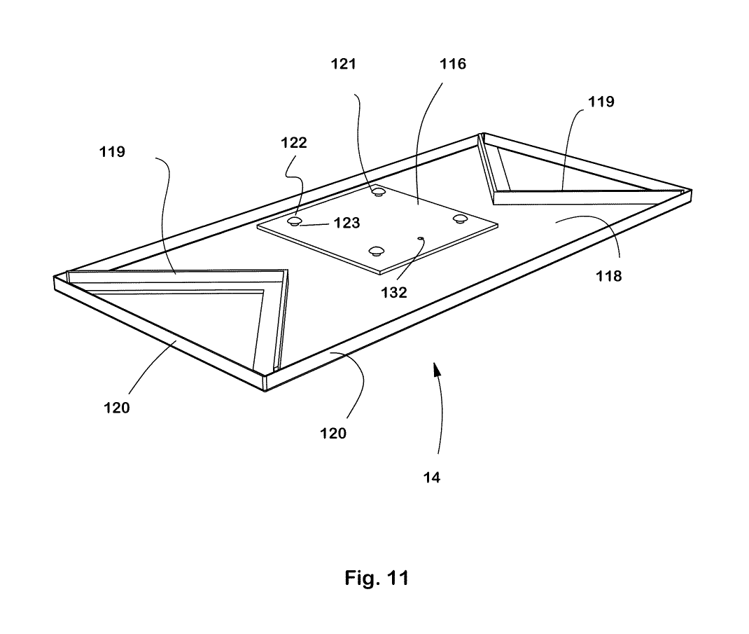

One of the tabletops is shown upside down in FIG. 11, to reveal a base plate 116 and a sheet metal panel 118 spot welded to the base plate. The panel has peripheral flanges 120 which reinforce the tabletop against bending. The base plate 116 reinforces the center of the panel, and pairs of reinforcing channel members 119, arranged in two vees, are spot welded to the panel to reinforce its outer portions against bending.

Four headed studs 121 are welded to the base plate, in a pattern corresponding to that of the keyhole apertures in the carriage. The heads 122 of the studs are sized to fit through large ends of the keyholes, and their necks 123 are sized to fit within the small ends of the keyholes, so the front table can be installed on the carriages simply by inserting the studs heads into the keyholes, and then moving the top laterally—in a horizontal direction perpendicular to the movement axes of the carriage—to lock the top to the carriage. A person cannot accidentally disengage the tabletop by leaning against the front end of the table, or by pushing or pulling on it: a deliberate lateral force is required.

The invention is subject to modifications and improvements. For example, one or more of the raised lips of the rear shelf and/or one or more of the raised lips of the front shelf may be replaced with a contoured shelf rod (not shown), which is raised approximately one-half inch from the shelf's planar surface so liquids can drain. Also, the tabletops may be labeled by being laser cut or embossed, e.g.: FR for front right, FL for front left, RR for rear right, and RL for rear left, to aid in assembly or reassembly of the table.

The examples given above are merely illustrative and are not meant to be an exhaustive list of all possible designs, embodiments, applications or modifications of the invention defined below.

We claim as our invention:

1. A surgical accessory table comprising
a structural base,
a first tabletop having a first top surface,
a second tabletop having a second top surface,
a height adjustment mechanism for raising or lowering said tabletops in unison, with respect to said base,
each tabletop being slidable along a first horizontal axis, with respect to said base, and
at least one of said tabletops being tiltable about a second horizontal axis perpendicular to said first horizontal axis, and
locking mechanisms for normally preventing tilting or sliding of said tabletops, said locking mechanisms being manually releasable to permit sliding and/or tilting of the tabletops.

2. The invention of claim 1, wherein said devices comprise, for each tabletop
a release handle which, when raised, permits the tabletop to be slid along said first axis, and
at least one release lever, which, when lifted, permits the tabletop to be tilted to a desired angle about said second axis.

3. The invention of claim 2, further comprising springs for biasing said mechanisms toward their locked positions.

4. The invention of claim 3, wherein said height adjustment mechanism comprises a pedal supported by the base, and linkage connecting said pedal to the height adjustment mechanism.

5. The invention of claim 1, wherein the stud fasteners are headed studs on one of the tabletop and the carriage, and further comprising corresponding plural keyhole apertures formed in one of the carriage and the tabletop.

6. The invention of claim 5, wherein each carriage has the keyhole apertures aligned all in the same direction, with their length perpendicular to the axis of movement of the respective carriage.

7. The invention of claim 6, wherein the keyhole apertures on one carriage are aligned in a direction opposite those of the keyhole apertures on the other carriage.

8. The invention of claim 1, wherein the tiltable tabletop is supported by a carriage, said carriage being mounted at its rearward end by hinge pins to a stationary cradle so that the carriage can be tilted from horizontal, and further comprising a tilt control mechanism for holding the carriage alternatively in a horizontal or tilted position, said tilt control mechanism comprising
a support rod passing beneath said carriage, said support rod being mounted on a member pivotally supported by a bracket attached to the cradle,
cam levers for pivoting said support rod and said member forward so as to allow the cradle to fall and pivot about the hinge pins to a tilted position, and
a linkage for manually activating each of said cam levers.

9. The invention of claim 8, wherein each of said levers has a pivot connection to said carriage and a horizontally extending pin proximate said pivot connection, and said linkage comprises a lever mounted on a horizontal rod supported by said carriage, said lever having an ear which can be lifted manually to cause the lever to engage said pin and cause a cam surface on the cam lever to drive said support rod to a position which allows the carriage to fall to said tilted position.

10. The invention of claim 8, wherein said carriage has an aperture therein, said member has a hook which protrudes through said slot and, in a first position of said hook, holds the table in its horizontal position, and, in a second position of said hook, releases the table, and further comprising a spring for biasing said hook toward said first position.
